(19) United States
(12) Patent Application Publication
(10) Pub. No.: US 2012/0289972 A1
(43) Pub. Date: Nov. 15, 2012

(54) HANDHELD DEVICE FOR STENT IMPLANTATION

(75) Inventors: Eugen Hofmann, Zuerich (CH); Raimund Moehl, Forch (CH)

(73) Assignee: BIOTRONIK AG, Buelach (CH)

(21) Appl. No.: 13/457,259

(22) Filed: Apr. 26, 2012

Related U.S. Application Data
(60) Provisional application No. 61/484,244, filed on May 10, 2011.

(51) Int. Cl. A61F 11/00

(52) U.S. Cl. 606/108

(57) ABSTRACT

A handheld device for the operation of an application system for stent implantation comprising an adapter for connection to an introducer sheath or a guide catheter, through which a wire or catheter can be inserted into a vessel. The adapter is in the form of a telescopic tube.

### CROSS REFERENCE TO RELATED APPLICATIONS

This invention claims benefit of priority to U.S. provisional patent application Ser. No. 61/484,244; the entire contents of which are herein incorporated by reference in their entirety.

### TECHNICAL FIELD

The invention relates to a handheld device for the operation of an application system for stent implantation comprising an adapter for connection to an introducer sheath or a guide catheter, through which a wire or catheter can be inserted into a vessel.

### BACKGROUND

Angioplasty, which is also referred to as percutaneous transluminal angioplasty (PTA), is a method for expanding or reopening constricted or obstructed blood vessels usually by balloon dilatation. In balloon dilatation within the scope of percutaneous transluminal angioplasty, a balloon installed on a vascular catheter is unfolded slowly and under pressure at the constricted vascular site. The constriction is thereby expanded such that it no longer obstructs blood flow, or does so to a lesser extent. Balloon catheters are nearly always inserted into the groin and placed in the stenosis (constriction) using a guide wire and guide catheter. In addition, stents are often implanted using a suitable system (stent implantation). A stent (which is also referred to as a vascular support) is a medical implant that is inserted into hollow organs, i.e. a blood vessel in this case, in order to hold it open. It is usually a small lattice framework in the shape of a small tube composed of metal or plastic.

To perform stent implantation, the first step typically is to insert a guide wire into the vessel via an introducer sheath or a guide catheter, and to advance the stent, which is disposed on a carrier system, to the site of the lesion using said guide wire. In many cases, the constriction is expanded using a balloon catheter (angioplasty) before the stent is inserted. The term “introducer sheath” in this case refers to hemostatic valves in general. They are usually thin tubes made of plastic, which include a valve mechanism that enables an interventional tool such as wires and catheters to be advanced, while simultaneously preventing blood from escaping. The stent—and therefore the vessel in the region of the lesion—is then dilated at the implantation site by expanding a balloon or, if a self-expanding stent is used, the stent is released by retracting an outer shaft.

While the stent is being placed, it must not be moved forward or backward in the axial direction as this could result in compression or extension of the stent. Such an axial deformation of the stent can result in the formation of cracks or the fracture of individual segments of the stent, which clearly increases the likelihood of a renewed vascular occlusion (restenosis).

### SUMMARY

The problem to be solved by the present invention is that of reducing or avoiding one or more disadvantages of the prior art. In particular, a problem addressed by the present invention is that of preventing compression or extension of the stent during dilatation.

The present invention solves this problem by providing a handheld device for the operation of an application system for stent implantation comprising an adapter for connection to an introducer sheath or a guide catheter, through which a wire or catheter can be inserted into a vessel. According to the invention, the adapter is designed as a telescopic tube.

A telescopic tube is composed of a plurality of tubes or cylinders (which may taper cylindrically and are parallel) guided within one another, which can be extended linearly to a maximum extension length until a stop is reached, and can then be retracted within one another in a space-saving manner. The telescopic tube is a technical extension of an arm in tubular form, which enables a desired distance to be attained in a variable manner. The position of the stent to be applied varies from patient to patient. The length of the telescopic tube, in the maximally extended state, should therefore be predefined such that the part of the stent carrier system extending out of the patient's body can be brought into a straight and elongated position. This length is composed of the useful length of the stent carrier system minus the length of the stent to be implanted and the length of the introducer sheath. For example, if the useful length of the stent carrier system is 135 cm, the stent length is 20 cm, and a length of the introducer sheath is 15 cm, the result is a length of 100 cm. This means that the telescopic tube must be extended to a length of at least 100 cm in order to ensure that a straight position of the stent carrier system is attained, if the objective is to implant the stent directly adjacent to the distal end of the introducer sheath. The further the stent is to be implanted in the body, the more the telescopic tube must be to shortened.

The telescopic tube preferably comprises 4 to 8 tubes or cylinders. The individual tubes or cylinders of the telescopic tube, in the maximally extended state, should preferably still be guided within one another along a length of at least 5 cm in order to ensure adequate stability.

The invention is therefore based on the idea that an unwanted axial deformation of the stent during expansion thereof can be prevented if movement of the handheld device in the axial direction is prevented in this phase of the operation. A special adapter is provided for this purpose, which is mounted on the handheld device and can be extended telescopically until it reaches the introducer sheath or the guide catheter. This adapter is in the form of a telescopic tube. The telescopic tube-like adapter is extended to the required length before the stent is expanded, and so the handheld device, in the straight and elongated position, is connected to the introducer sheath or the guide catheter. It can therefore be ensured that unwanted compression or extension of the stent is prevented. The adapter can be designed to be securely affixed to the handheld device, or it can be designed as a separable additional part. The adapter can be composed of metal, e.g. surgical steel.

The adapter preferably comprises locking elements that can be used to immobilize a length of the telescopic tube. If the adapter is then extended to the desired length, the individual tubes can be locked in position by one turn of rotation, for example. Suitable locking elements include complementary notches and ridges on the tubes or clamping elements, such as levers or the like.

Another aspect of the invention is that of providing an application system for stent implantation, which comprises the previously described handheld device.

### DESCRIPTION OF THE DRAWINGS

The invention is explained in greater detail below with reference to embodiments and the to related figures. In the figures:

FIG. 1 shows components of an application system for stent implantation comprising a handheld device having an adapter according to the invention.

FIG. 2 shows the adapter according to the invention, in an enlarged depiction.

FIG. 3 is used to illustrate the use of the application system according to the invention within the scope of stent implantation.

### DETAILED DESCRIPTION

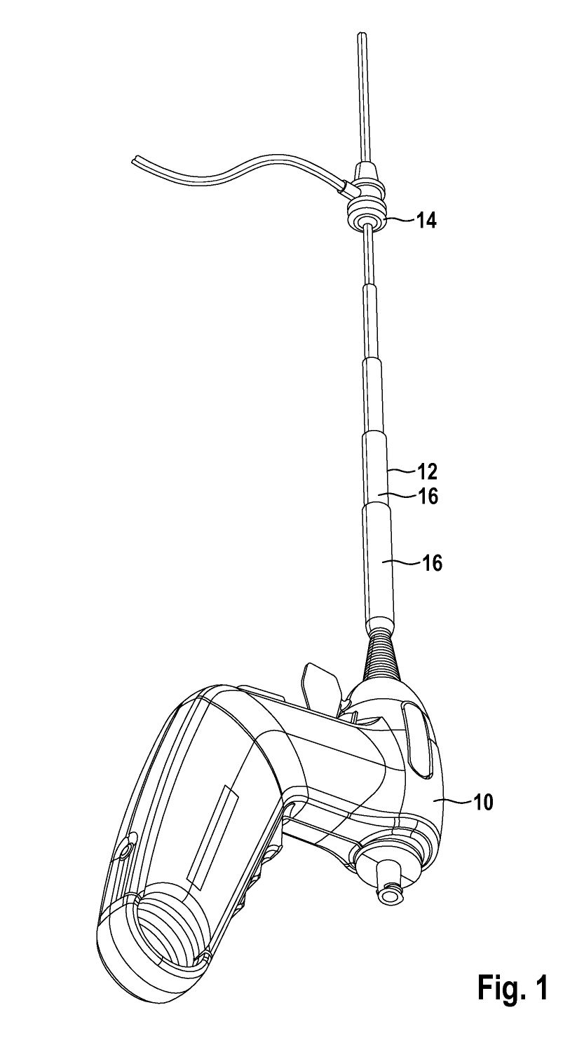

FIG. 1 shows a handheld device 10 of the type used for stent implantation. Handheld device 10 is connected to an introducer sheath 14 via an adapter 12. Using adapter 12, guide wires and a stent carrier system can be inserted during the interventional procedure.

Adapter 12 is characterized in that it is designed as a telescopic tube, i.e. in this case it is composed of a plurality of tubes 16 which taper cylindrically, are guided in parallel within one another, and can be extended linearly to a maximum extension length until a stop is reached, and can then be retracted within one another in a space-saving manner. Adapter 12 can be composed of medical steel, for example.

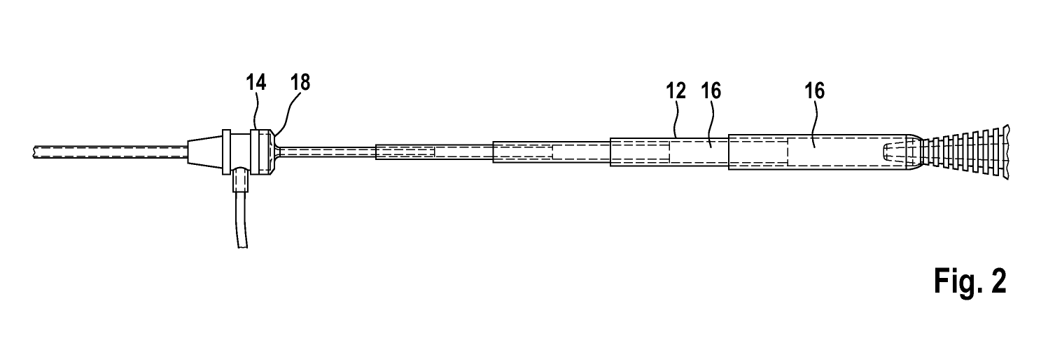

FIG. 2 shows adapter 12 once more, in an enlarged depiction. In this case, individual tubes 16 have been retracted inward by approximately one-third of the maximum extension length, as an example. Adapter 12 comprises an end 18 having a complementary design for establishing an interference fit at introducer sheath 14.

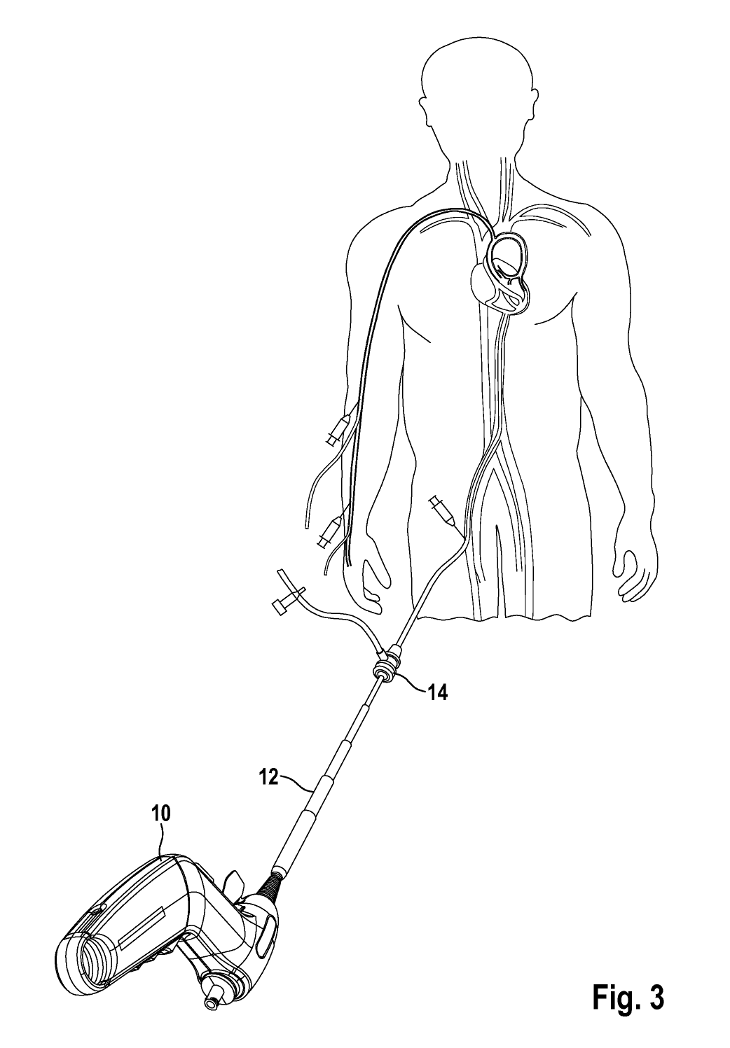

FIG. 3 is a schematic illustration of the use of the application system within the scope of stent implantation. After vasopuncture in the lumbar region, an introducer sheath 14 is placed, and handheld device 10 comprising telescopic tube-type adapter 12 is connected thereto. Within the scope of percutaneous transluminal coronary angioplasty (PTCA), a stent application system, among other things, is guided through introducer sheath 14 to the heart. In the' region of the stenosis, an outer tube is retracted from the stent application system, thereby simultaneously releasing the stent. Before this step, adapter 12 is extended so far that any axial movement of handheld device 10 initially results only in a change in length of adapter 12, thereby enabling compression or extension of the stent to be prevented in this phase.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A handheld device for the operation of an application system for stent implantation comprising an adapter for connection to an introducer sheath or a guide catheter, through which a wire or catheter can be inserted into a vessel, the adapter being designed as a telescopic tube.

2. The handheld device according to claim 1, wherein the telescopic tube comprises 4 to 8 tubes or cylinders.

3. The handheld device according to claim 1, wherein the individual tubes or cylinders of the telescopic tube, in the maximally extended state, remain guided within one another along a length of at least 5 cm.

4. The handheld device according to claim 1, wherein the adapter comprises locking elements for immobilizing a length of the telescopic tube.

5. An application system for stent implantation, comprising a handheld device according to claim 1.
